(19) United States
(12) Patent Application Publication
(10) Pub. No.: US 2005/0248752 A1
(43) Pub. Date: Nov. 10, 2005

(54) Solar rating system for intraocular lens implants

(76) Inventor: Gary W. Hall, Paradise Valley, AZ (US)

Correspondence Address: MICHAEL WINFIELD GOLTRY, 4000 N. CENTRAL AVENUE, SUITE 1220, PHOENIX, AZ, 85012, US

(21) Appl. No.: 11/118,812

(22) Filed: Apr. 29, 2005

Related U.S. Application Data
(60) Provisional application No. 60/567,175, filed on Apr. 30, 2004.

(51) Int. Cl. G01N 21/00; A61F 2/16

(52) U.S. Cl. 356/124; 623/912; 356/432

(57) ABSTRACT

A method of rating an intraocular lens (IOL), includes providing an IOL, measuring a retinal toxicity level for a plurality of waveband increments of environmental solar radiation between approximately 300 nm and approximately 515 nm transmitted through the IOL to provide a plurality of measured retinal toxicity level values, averaging the plurality of measured retinal toxicity level values to provide an average retinal toxicity level value, transforming the average retinal toxicity level value to a rating value, and informing a prospective consumer of the rating value.

### CROSS-REFERENCE TO RELATED APPLICATION

This application claims the benefit of U.S. Provisional Patent Application Ser. No. 60/567,175, filed Apr. 30, 2004.

### FIELD OF THE INVENTION

The present invention relates to intraocular lens implants and, more particularly, a method of rating the solar protection properties of intraocular lens implants.

### BACKGROUND OF THE INVENTION

An intraocular lens (IOL) is an implanted lens in the eye, usually replacing the existing crystalline lens because it has been clouded over by a cataract. They usually consist of a plastic lens with plastic side struts called haptics to hold the lens in place within the capsular bag.

Insertion of an intraocular lens is the most commonly performed eye surgical procedure, and cataracts are the most common eye disease. The procedure can be done under local anesthesia with the patient awake throughout the operation which usually takes less than 30 minutes in the hands of an experienced ophthalmologist.

There are foldable intraocular lenses made of acrylic or silicone that can be rolled up and inserted through a tube with a very small incision not requiring any stitches, and inflexible lenses, typically made of polymethyl methacrylate, requiring a larger incision.

Unlike the natural lens, the curvature of a conventional intraocular lens cannot be changed by the eye. Standard intraocular lenses provide good distance vision and the patient needs reading glasses for near vision. Newer bifocal intraocular lenses give distance vision in one area and near vision in another area of the vision field.

Optical Radiation Corporation introduced the first IOL containing an ultraviolet (UV) light-blocking chromophore in the mid-1980's. As a result of this development, IOLs were no longer considered just an optical device, but a protective device as well. Subsequently, with an increasing awareness of UV retinal phototoxicity, most of the present IOLs contain a chromophore that attenuates UV radiation.

Ultraviolet radiation is toxic to virtually every part of the human eye. Because UV radiation does not contribute to human vision, every effort should be made through protective devices, such as sunglasses and IOLs, to prevent this radiation from reaching the eye and lids.

It is well understood by those having ordinary skill that retinal toxicity is produced in response to exposure of the retina to intense short wavelength visible radiation. Moreover, it has been noticed that prolonged exposure to lower intensity blue light can cause the onset of macular degeneration.

The blue light-blocking IOL, which is an IOL capable of attenuating a larger portion of the blue light spectrum, was first introduced in 2003. Because blue light is part of the visible spectrum, and because of the inconsistent results of edemological studies linking chronic blue light exposure to macular degeneration, the introduction of the blue light-blocking IOL has been controversial.

In addition to improving visual function through advancements in optics, providing retinal protection is becoming an increasingly recognizable function of the IOL. To date, however, there has been an unmet need to provide surgeons and users of IOLs with accurate information about the retinal protective properties of IOLs for allowing surgeons and consumers of the IOLs to be better informed regarding the solar protection qualities of IOLs.

### SUMMARY OF THE INVENTION

According to the invention, a method of rating an intraocular lens (IOL) includes providing an IOL, measuring a retinal toxicity level for a plurality of waveband increments of environmental solar radiation between approximately 300 nm and approximately 515 nm transmitted through the IOL to provide a plurality of measured retinal toxicity level values, averaging the plurality of measured retinal toxicity level values to provide an average retinal toxicity level value, transforming the average retinal toxicity level value to a rating value, and informing a prospective consumer of the rating value. Measuring the retinal toxicity level for the plurality of waveband increments of environmental solar radiation between approximately 300 nm and approximately 515 nm transmitted through the IOL further includes measuring the retinal toxicity level for each 5 nm waveband increment of environmental solar radiation between approximately 300 nm and approximately 515 nm transmitted through the IOL. Informing a prospective consumer of the rating value consists of associating the rating value with the IOL, such as by placing the rating value on a label applied to the IOL and/or affixing the rating value to packaging for the IOL.

According to the invention, another method of rating an intraocular lens (IOL) includes providing an IOL, measuring a first retinal toxicity level for a plurality of waveband increments of environmental solar radiation between approximately 300 nm and approximately 400 nm transmitted through the IOL to provide a first plurality of measured retinal toxicity level values, averaging the first plurality of measured retinal toxicity level values to provide a first average retinal toxicity level value, transforming the first average retinal toxicity level value to a first rating value, and informing a prospective consumer of the first rating value. Measuring the first retinal toxicity level for the plurality of waveband increments of environmental solar radiation between approximately 300 nm and approximately 400 nm transmitted through the IOL further includes measuring the first retinal toxicity level for each 5 nm waveband increment of environmental solar radiation between approximately 300 nm and approximately 400 nm transmitted through the IOL. Informing a prospective consumer of the first rating value consists of associating the first rating value with the IOL, such as by placing the first rating value on a label applied to the IOL and/or affixing the first rating value to packaging for the IOL. The method still further includes measuring a second retinal toxicity level for a plurality of waveband increments of environmental solar radiation between approximately 400 nm and approximately 515 nm transmitted through the IOL to provide a second plurality of measured retinal toxicity level values, averaging the second plurality of measured retinal toxicity level values to provide a second average retinal toxicity level value, transforming the second average retinal toxicity level value to a second rating value, and informing a prospective consumer of the second rating value. Measuring the second retinal toxicity level for the plurality of waveband increments of environmental solar radiation between approximately 400 nm and approximately 515 nm transmitted through the IOL further includes measuring the second retinal toxicity level for each 5 nm waveband increment of environmental solar radiation between approximately 400 nm and approximately 515 nm transmitted through the IOL. Informing the prospective consumer of the second rating value consists of associating the second rating value with the IOL, such as placing the second rating value on a label applied to the IOL and/or affixing the second rating value to packaging for the IOL. Still further to the present method are the steps of averaging the first average retinal toxicity level and the second average retinal toxicity level value to provide a total average retinal toxicity level, transforming the total average retinal toxicity level value to a third rating value, and informing a prospective consumer of the third rating value. Furthermore, the step of informing the prospective consumer of the third rating value further comprises associating the third rating value with the IOL, such as placing the third rating value on a label applied to the IOL and/or affixing the third rating value to packaging for the IOL.

According to the invention, yet another method of rating an intraocular lens (IOL) includes providing an IOL, measuring a first retinal toxicity level for a plurality of waveband increments of environmental solar radiation between approximately 400 nm and approximately 515 nm transmitted through the IOL to provide a first plurality of measured retinal toxicity level values, averaging the first plurality of measured retinal toxicity level values to provide a first average retinal toxicity level value, transforming the first average retinal toxicity level value to a first rating value, and informing a prospective consumer of the first rating value. Measuring the first retinal toxicity level for the plurality of waveband increments of environmental solar radiation between approximately 400 nm and approximately 515 nm transmitted through the IOL further includes measuring the first retinal toxicity level for each 5 nm waveband increment of environmental solar radiation between approximately 400 nm and approximately 515 nm transmitted through the IOL. Informing a prospective consumer of the first rating value consists of associating the first rating value with the IOL, such as by placing the first rating value on a label applied to the IOL and/or affixing the first rating value to packaging for the IOL. The method still further includes measuring a second retinal toxicity level for a plurality of waveband increments of environmental solar radiation between approximately 300 nm and approximately 400 nm transmitted through the IOL to provide a second plurality of measured retinal toxicity level values, averaging the second plurality of measured retinal toxicity level values to provide a second average retinal toxicity level value, transforming the second average retinal toxicity level value to a second rating value, and informing a prospective consumer of the second rating value. Measuring the second retinal toxicity level for the plurality of waveband increments of environmental solar radiation between approximately 300 nm and approximately 400 nm transmitted through the IOL further includes measuring the second retinal toxicity level for each 5 nm waveband increment of environmental solar radiation between approximately 300 nm and approximately 400 nm transmitted through the IOL. Informing the prospective consumer of the second rating value consists of associating the second rating value with the IOL, such as placing the second rating value on a label applied to the IOL and/or affixing the second rating value to packaging for the IOL. Still further to the present method are the steps of averaging the first average retinal toxicity level and the second average retinal toxicity level value to provide a total average retinal toxicity level, transforming the total average retinal toxicity level value to a third rating value, and informing a prospective consumer of the third rating value. Furthermore, the step of informing the prospective consumer of the third rating value further comprises associating the third rating value with the IOL, such as placing the third rating value on a label applied to the IOL and/or affixing the third rating value to packaging for the IOL.

Consistent with the foregoing summary of preferred embodiments, and the ensuing detailed description, which are to be taken together, the invention also contemplates associated method embodiments.

### DETAILED DESCRIPTION OF A PREFERRED EMBODIMENT

Since it's inception in the 1970's, there has been nearly worldwide adoption of the sun protection factor (SPF) system for rating commercial sunscreen products' protection against erythema or sunburned skin. The SPF values range from 0 to 50 and represent multiples of the time required to reach erythema compared to unprotected skin. For example, an SPF of 15 indicates that it would require fifteen times longer to reach the level of sunburn than without any sunscreen. Although tremendous variations exist in an individual's natural susceptibility to skin cancer or sunburn, based on such differences as skin pigmentation, immune systems, and environmental exposure, the SPF system has become internationally recognized. The SPF system not only allows consumers to compare the protective qualities of commercial sunscreen products, it also has heightened public awareness of the increasing incidence of skin cancer occurring worldwide. Developing such a rating system for IOLs, however, is problematic and much more difficult than the SPF system because the phototoxicity of the retina (the most important tissue protected by an IOL is caused not only by exposure to UV light spectrum but also a portion of the visible light spectrum.

Disclosed is a method for rating the solar protective properties of an intraocular lens (IOL). The method provides the user or consumer of the IOL with valuable information concerning the value or rating of the solar protective properties of the IOL in the form of a value called a retinal protection factor (RPF). The user or consumer is able to use the RPF to make an informed decision when using or purchasing an IOL rated according to the method of the invention. The RPF represents the solar protective property of an IOL that combines separate values for blue/violet radiation and for ultraviolet (UV) radiation. The inclusion of the RPF with an IOL will allow surgeons and consumers to be better informed about the IOL, and may also serve to increase public awareness about ocular solar phototoxicity.

Unlike conventional rating systems for sunglasses, the IOL rating method disclosed herein is unique because the phototoxicity of the retina includes UV and a portion of the visible spectrum. Since the retina is sensitive to radiation exposure, the eye has developed its own protective mechanism, primarily through the cornea and the lens, to filter out potentially harmful sunrays. When removing a cataract and replacing it with an IOL, there must, therefore, be an awareness of the optical qualities of the IOL and the retina's inherent protective characteristics.

To rate an IOL and arrive at an RPF value for an IOL according to the principle of the invention, the action of spectral curves is considered, which is the amount of energy required to cause a visible, permanent lesion in twenty-four to forty-eight hours after acute exposure. The average of the retinal sensitivity in five nanometer waveband increments over the UV spectra (315 nm-400 nm) and the blue/violet spectra (400 nm-515 nm) shows that the retina is approximately five times more sensitive to the UV_{A} spectra than the blue/violet spectra. The environmental radiation toxicity to the retina for a specified waveband is the product of its sensitivity and the amount of energy reaching the retina. The amount of energy reaching the retina is complicated by the tremendous variations of environmental solar energy occurring daily, which is based on time, season, location, weather, pollution, and other factors. In addition, the quantity of solar energy reaching the retina varies with the square of the pupil diameter, the direction of gaze, the reflection of the surrounding surfaces, and changes throughout life with the increasing filtration properties of the human lens. Accordingly, in a preferred method for rating an IOL and providing an RPF value for an IOL being reflective of the rating, an approximation of peak solar energy values at sea level is used. Furthermore, after a human lens is removed and replaced with an IOL, the solar energy reaching the retina is the product of the environmental solar energy, transmittance to the anterior surface of the pseudophakos, and the transmittance through the IOL.

Evaluation of the transmittance of solar energy to the adult human retina shows that there is approximately twice the amount of the blue/violet spectral light in the environment as UV_{A} spectral radiation. Also, an average of nearly 60% of the blue/violet spectral light reaches the retina, whereas an average of only 1.3% of UV_{A} spectral radiation reaches the retina. When an average of the retinal toxicity levels over the blue/violet spectral range and the UV_{A} spectral range is taken, it is found that blue/violet radiation is approximately fourteen times more toxic than UV_{A} radiation, with the peak toxicity level occurring at approximately 440 nm.

According to the principle of the invention, a method of rating an IOL and determining an RPF value corresponding to the solar protection properties of the IOL begins with establishing a retinal toxicity level (RTLλ) for each 5 nm waveband of environmental solar radiation between 300 nm and 515 nm transmitted through the IOL to be rated. Solar radiation falling outside of 300 nm-515 nm is not considered because solar radiation below 300 nm does not transmit to the human lens or pseudophakos, and the incidence of retinal photochemical toxicity as a result of exposure of the retina to solar radiation above 515 nm is negligible. The RTLλ determined at each 5 nm waveband between 300 nm and 515 nm is then averaged to provide an average RTLλ (˜RTLλ) value for the IOL between 300 nm and 515 nm, which is an average of the RTLλ values for UV radiation (300 nm-400 nm) and blue/violet radiation (400 nm-515 nm).

According to the invention, RTLλ is the product of retinal sensitivity (Sλ), peak solar energy (Eλ) in the environment, and transmittance to the retina (T_{R}λ) through the IOL being rated, in which RTLλ=(Sλ) (Eλ) (T_{R}λ), Sλ is the inverse of the action spectrum and the quantum of energy reaching the retina within a specified waveband over a predetermined period of time. The peak quantum of energy of each waveband reaching the retina is the product of Eλ and T_{R}λ. There are many published sources of Eλ values, and the preferred embodiment set forth herein uses established Eλ values taken at 5 nm intervals between 300 nm-515 nm at sea level as published in Hynek J A, ed.; Astophysics: a topical symposium commemorating the 50^{th} anniversary of the Yerkes Observatory and a half-century of progress in astrophysics; New York, McGraw-Hill Book Company, 1951. Like Eλ values, there are many published sources of Sλ values, and the preferred method set forth herein uses Sλ values based on 100-second exposure data taken at 5 nm intervals between 300 nm-515 nm as published in Pitts D G and Kleinstein R N, eds.; Environmental Vision: interactions of the eye, vision, and the environment; Boston, Butterworth-Heinemann, 1993:185.

The T_{R}λ values are broken down segmentally from the anterior surface of the lens, from the lens to the vitreous, and from the vitreous to the retina. In order to determine the percent transmittance of radiation to the retina in the pseudophakic eye, the product of the transmittance to the anterior portion of the human lens, Tlensλ, and the transmittance through the IOL, Tλ, is used for each IOL rated, in which T_{R}λ=(Tlensλ) (Tλ) at each 5 nm increment from 300 nm-515 nm. The total amount of UV radiation from 300 nm-515 nm transmitted through the IOL is the basis for rating the solar protection properties of the IOL. The transmittance data for the tested IOLs was derived from a Schimadzu spectrophotometer, model no. UV-1601PC, and the transmission studies used for the human lens were taken from the work of Lerman as published in Lerman S. Light-Induced Changes in Ocular Tissues, Miller D, ed.; Clinical Light Damage to the Eye; New York, Springer-verlag, 1987:183-215.

An RPF value for the rated IOL for the UV spectra (300 nm-400 nm) is derived taking ˜RTLλ value for the UV spectra (300 nm-400 nm) and deducting the ˜RTLλ value from 100. Although the RPF is the total average retinal toxicity level for both UV_{A} and blue/violet less 100, the two individual components for UV_{A} and blue/violet factors can be represented separately as RPFᵤᵥ and RPF_{blue/violet} values, in which the RPFᵤᵥ value for the rated IOL for the UV spectra (300 nm-400 nm) is derived taking ˜RTLλ value for the UV spectra (300 nm-400 nm) and deducting the ˜RTLλ value for the UV spectra from 100, and the RPF_{blue/violet} value for the rated IOL for the blue/violet spectra (400 nm-515 nm) is derived taking ˜RTLλ value for the blue/violet spectra (400 nm-515 nm) and deducting the ˜RTLλ value for the blue/violet spectra from 100.

And so the present invention is a rating system to incorporate RPF values for an IOL. One value is assigned to UV radiation, namely, the RPFᵤᵥ, another value is assigned to blue/violet light, namely, the RPF_{blue/violet}, and a third value is assigned to the UV spectra including both UV radiation and blue/violet radiation, namely, the RPF. RPFᵤᵥ represents the solar protection property of the tested IOL for UV radiation (300 nm-400 nm), RPF_{blue/violet} represents the solar protection property of the tested IOL for blue/violet radiation (400 nm-515 nm), and RPF which represents the solar protection property of the tested IOL for UV radiation and blue/violet radiation (300 nm-515 nm).

The total amount of UV radiation (300 nm-400 nm) transmitted through the IOL serves as the basis for rating the total UV protection of the IOL. The total amount of blue/violet radiation (400 nm-515 nm) transmitted through the IOL serves as the basis for rating the total blue/violet protection of the IOL. In this rating system, the retinal toxicity levels measured at 5 nm intervals from 300 nm to 515 nm are averaged and then subtracted from 100 to reach the rating for UV and blue/violet protection as expressed by the RPF value. The retinal toxicity levels measured at 5 nm intervals from 300 nm to 400 nm are averaged and then subtracted from 100 to reach the rating for UV protection as expressed by the RPFᵤᵥ value. Further, the retinal toxicity levels measured at 5 nm intervals from 400 nm to 515 nm are averaged and then subtracted from 100 to reach the rating for UV_{blue/violet} protection as expressed by the RPF_{blue/violet} value.

The RPF values fall between 0 and 100. An RPF of 0 represents 100% transmission of UV and blue/violet radiation to the eye through the IOL, while a rating of 100 represents 100% absorption by the IOL and 0% transmission to the eye. Therefore, by looking at the rating, the potential eye protection of an IOL rated according to the principles of the invention can be readily determined.

The IOL rating expressed as an RPF value, once measured for a particular IOL, is to be associated with the IOL and should, for instance be available to the consumer at the point of sale, in accordance with the principle of the invention. Informing the prospective consumer of the RPF preferably includes associating the RPF with the IOL, such as placing the RPF value on a label applied to the IOL and/or affixing the RPF value to packaging for the IOL. Preferably, the RPF, the RPFᵤᵥ, and the RPF_{blue/violet} will be associated with the IOL and be available to the customer at the point of sale for providing the most information concerning the solar protection properties of the IOL. However, only one of the RPF, the RPFᵤᵥ, and the RPF_{blue/violet} may be used, or a selected combination of these RPF values.

The present invention is described above with reference to a preferred embodiment. However, those skilled in the art will recognize that changes and modifications may be made in the described embodiment without departing from the nature and scope of the present invention. Various changes and modifications to the embodiment herein chosen for purposes of illustration will readily occur to those skilled in the art. To the extent that such modifications and variations do not depart from the spirit of the invention, they are intended to be included within the scope thereof.

1. A method of rating an intraocular lens (IOL), comprising steps of:
providing an IOL;
measuring a retinal toxicity level for a plurality of waveband increments of environmental solar radiation between approximately 300 nm and approximately 515 nm transmitted through the IOL to provide a plurality of measured retinal toxicity level values;
averaging the plurality of measured retinal toxicity level values to provide an average retinal toxicity level value;
transforming the average retinal toxicity level value to a rating value; and
informing a prospective consumer of the rating value.

2. The method according to claim 1, wherein the step of measuring the retinal toxicity level for the plurality of waveband increments of environmental solar radiation between approximately 300 nm and approximately 515 nm transmitted through the IOL further comprises measuring the retinal toxicity level for each 5 nm waveband increment of environmental solar radiation between approximately 300 nm and approximately 515 nm transmitted through the IOL.

3. The method according to claim 1, wherein the step of informing a prospective consumer of the rating value further comprises associating the rating value with the IOL.

4. A method of rating an intraocular lens (IOL), comprising steps of:
providing an IOL;
measuring a first retinal toxicity level for a plurality of waveband increments of environmental solar radiation between approximately 300 nm and approximately 400 nm transmitted through the IOL to provide a first plurality of measured retinal toxicity level values;
averaging the first plurality of measured retinal toxicity level values to provide a first average retinal toxicity level value;
transforming the first average retinal toxicity level value to a first rating value; and
informing a prospective consumer of the first rating value.

5. The method according to claim 4, wherein the step measuring the first retinal toxicity level for the plurality of waveband increments of environmental solar radiation between approximately 300 nm and approximately 400 nm transmitted through the IOL further comprises measuring the first retinal toxicity level for each 5 nm waveband increment of environmental solar radiation between approximately 300 nm and approximately 400 nm transmitted through the IOL.

6. The method according to claim 4, wherein the step of informing a prospective consumer of the first rating value further comprises associating the first rating value with the IOL.

7. The method according to claim 6, further comprising:
measuring a second retinal toxicity level for a plurality of waveband increments of environmental solar radiation between approximately 400 nm and approximately 515 nm transmitted through the IOL to provide a second plurality of measured retinal toxicity level values;
averaging the second plurality of measured retinal toxicity level values to provide a second average retinal toxicity level value;
transforming the second average retinal toxicity level value to a second rating value; and
informing a prospective consumer of the second rating value.

8. The method according to claim 7, wherein the step measuring the second retinal toxicity level for the plurality of waveband increments of environmental solar radiation between approximately 400 nm and approximately 515 nm transmitted through the IOL further comprises measuring the second retinal toxicity level for each 5 nm waveband increment of environmental solar radiation between approximately 400 nm and approximately 515 nm transmitted through the IOL.

9. The method according to claim 8, wherein the step of informing the prospective consumer of the second rating value further comprises associating the second rating value with the IOL.

10. The method according to claim 9, further comprising:
averaging the first average retinal toxicity level and the second average retinal toxicity level value to provide a total average retinal toxicity level;
transforming the total average retinal toxicity level value to a third rating value; and
informing a prospective consumer of the third rating value.

11. The method according to claim 10, wherein the step of informing the prospective consumer of the third rating value further comprises associating the third rating value with the IOL.

12. A method of rating an intraocular lens (IOL), comprising steps of:
providing an IOL;
measuring a first retinal toxicity level for a plurality of waveband increments of environmental solar radiation between approximately 400 nm and approximately 515 nm transmitted through the IOL to provide a first plurality of measured retinal toxicity level values;
averaging the first plurality of measured retinal toxicity level values to provide a first average retinal toxicity level value;
transforming the first average retinal toxicity level value to a first rating value; and
informing a prospective consumer of the first rating value.

13. The method according to claim 12, wherein the step measuring the first retinal toxicity level for the plurality of waveband increments of environmental solar radiation between approximately 400 nm and approximately 515 nm transmitted through the IOL further comprises measuring the first retinal toxicity level for each 5 nm waveband increment of environmental solar radiation between approximately 400 nm and approximately 515 nm transmitted through the IOL.

14. The method according to claim 12, wherein the step of informing a prospective consumer of the first rating value further comprises associating the first rating value with the IOL.

15. The method according to claim 14, further comprising:
measuring a second retinal toxicity level for a plurality of waveband increments of environmental solar radiation between approximately 300 nm and approximately 400 nm transmitted through the IOL to provide a second plurality of measured retinal toxicity level values;
averaging the second plurality of measured retinal toxicity level values to provide a second average retinal toxicity level value;
transforming the second average retinal toxicity level value to a second rating value; and
informing a prospective consumer of the second rating value.

16. The method according to claim 15, wherein the step measuring the second retinal toxicity level for the plurality of waveband increments of environmental solar radiation between approximately 300 nm and approximately 400 nm transmitted through the IOL further comprises measuring the second retinal toxicity level for each 5 nm waveband increment of environmental solar radiation between approximately 300 nm and approximately 400 nm transmitted through the IOL.

17. The method according to claim 15, wherein the step of informing the prospective consumer of the second rating value further comprises associating the second rating value with the IOL.

18. The method according to claim 17, further comprising:
averaging the first average retinal toxicity level and the second average retinal toxicity level value to provide a total average retinal toxicity level;
transforming the total average retinal toxicity level value to a third rating value; and
informing a prospective consumer of the third rating value.

19. The method according to claim 18, wherein the step of informing the prospective consumer of the third rating value further comprises associating the third rating value with the IOL.
